(19) United States
(12) Patent Application Publication
(10) Pub. No.: US 2008/0064926 A1
(43) Pub. Date: Mar. 13, 2008

(54) Laryngoscope with wireless image transmission

(76) Inventor: Tien-Sheng Chen, Taipei (TW)

Correspondence Address: Hershkovitz & Associates, LLC, 2845 Duke Street, Alexandria, VA, 22314, US

(21) Appl. No.: 11/600,165

(22) Filed: Nov. 16, 2006

(30) Foreign Application Priority Data
Sep. 13, 2006 (TW) 095216314

(51) Int. Cl. A61B 1/04

(52) U.S. Cl. 600/110

(57) ABSTRACT

A laryngoscope for wireless image transmission has a handle and a blade. The handle has a power device and a terminal connecting to the power device. The blade is mounted detachably on the handle and has a camera with LED and a contact connecting to each other. A wireless transmitter connects to the camera with LED to send the image. The blade is inserted into the mouth of the patient to lean against the patient's tongue. The throat can be lighted by the camera with LED and the image can be transferred wirelessly to a screen.

### BACKGROUND OF THE INVENTION

1. Field of the Invention

The present invention relates to a laryngoscope with wireless image transmission, and more specifically to a laryngoscope which can transmit image without using wires.

2. Description of the Related Art

A plastic endotracheal tube should be inserted into the patient's trachea to maintain breathing of the patient. During the insertion of the plastic endotracheal tube into the trachea via a conventional laryngoscope, difficulty is encountered in about 20% of patients due to their short chins or stiff necks. Hence, a lens is mounted in the front of the first conventional laryngoscope and connected to a screen. However, the doctor will pay more attention to the screen and less to the plastic endotracheal tube. This way, the doctor's hands and eyes cannot coordinate smoothly. Furthermore, the first conventional laryngoscope is difficult to carry due to its large bulk, and the blade portion is not easily sterilized.

A second conventional laryngoscope has a plate, a lens and a light source mounted on the plate. However, the lens has a limited angle and the light source is too big for the doctor to operate. Furthermore, the doctor's hand and eyes cannot coordinate smoothly.

A third conventional laryngoscope has a handle and a plate mounted with a light source and a camera. The handle has an inbuilt electrical power, while the plate connected to the handle which is detachable. The plate is pressed against the patient's tongue to expose the guttural condition which can then be transferred to a screen by the camera. However, due to its big bulk, the third conventional laryngoscope is difficult to carry.

The invention provides a portable laryngoscope with wireless image transmission to mitigate or obviate the aforementioned problems.

### SUMMARY OF THE INVENTION

The main objective of the present invention is to provide a laryngoscope that can transmit images without using wires.

Other objectives, advantages and novel features of the invention will become more apparent from the following detailed descriptions in conjunction with the accompanying drawings.

### BRIEF DESCRIPTION OF THE DRAWINGS

FIG. 1 is a perspective view of a laryngoscope with wireless image transmission in accordance with the present invention;

FIG. 2 is an exploded perspective view of the laryngoscope with wireless image transmission in FIG. 1;

FIG. 3 is a perspective view of a laryngoscope with wireless image transmission in accordance with the present invention;

FIG. 4 is an operational side view of the laryngoscope with wireless image transmission in FIG. 1; and

FIG. 5 is a block diagram of the laryngoscope with wireless image transmission in FIG. 1.

### DETAILED DESCRIPTION OF THE PREFERRED EMBODIMENT

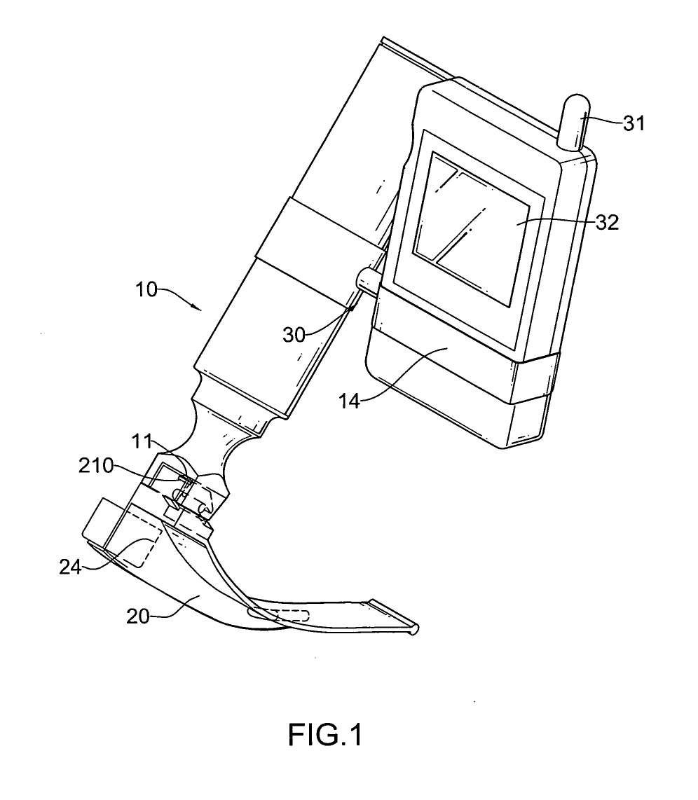

With reference to FIG. 1, a laryngoscope with wireless image transmission in accordance with the present invention has a handle (10), a blade (20) and a display (30).

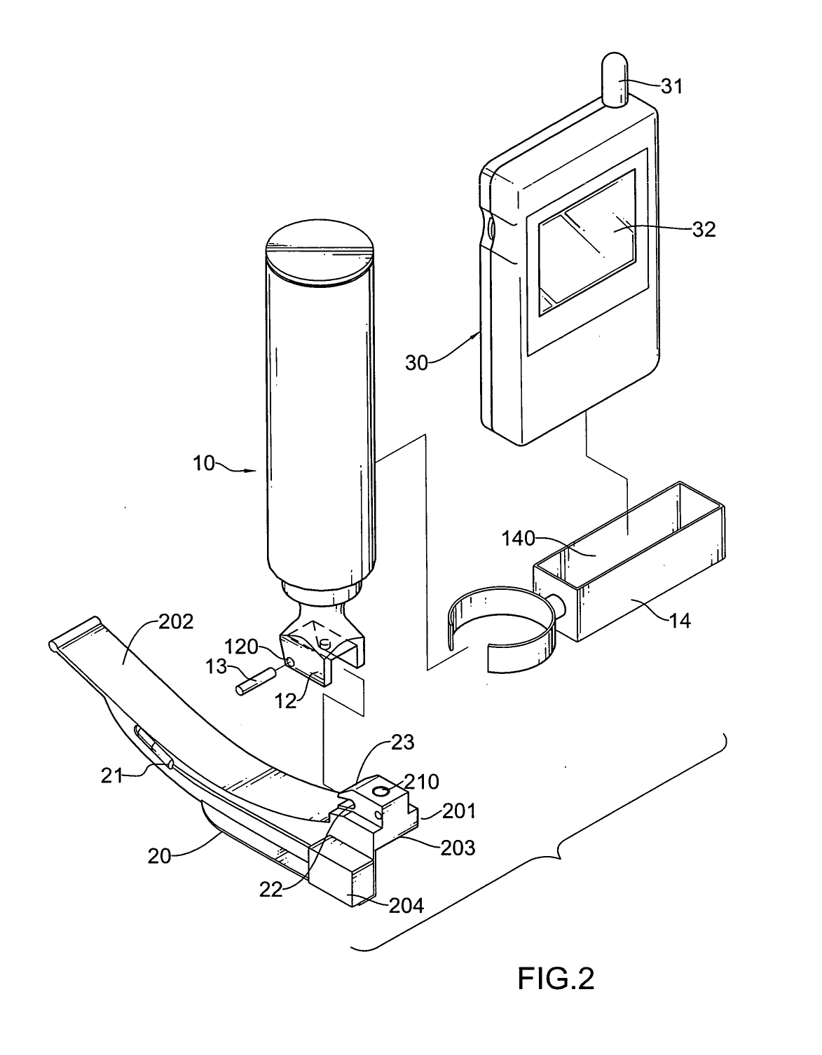

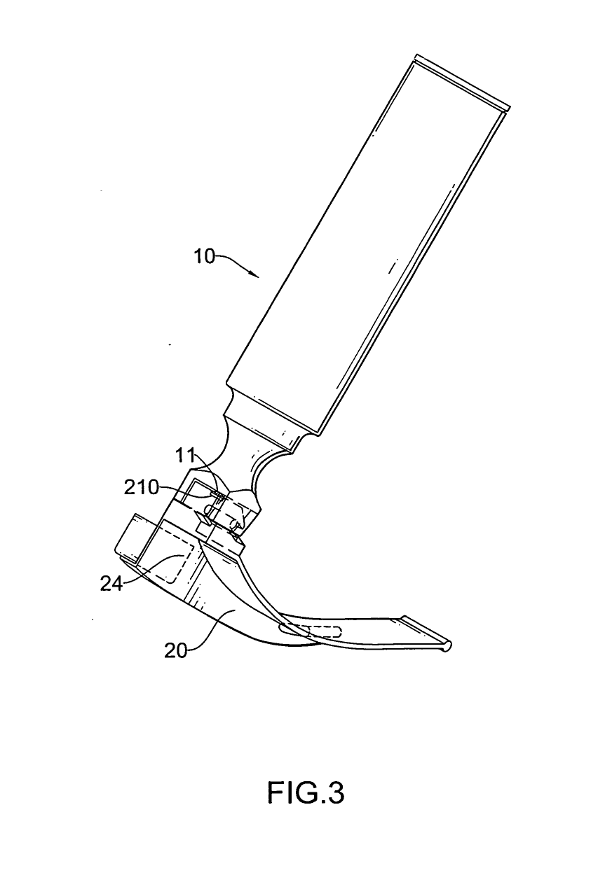

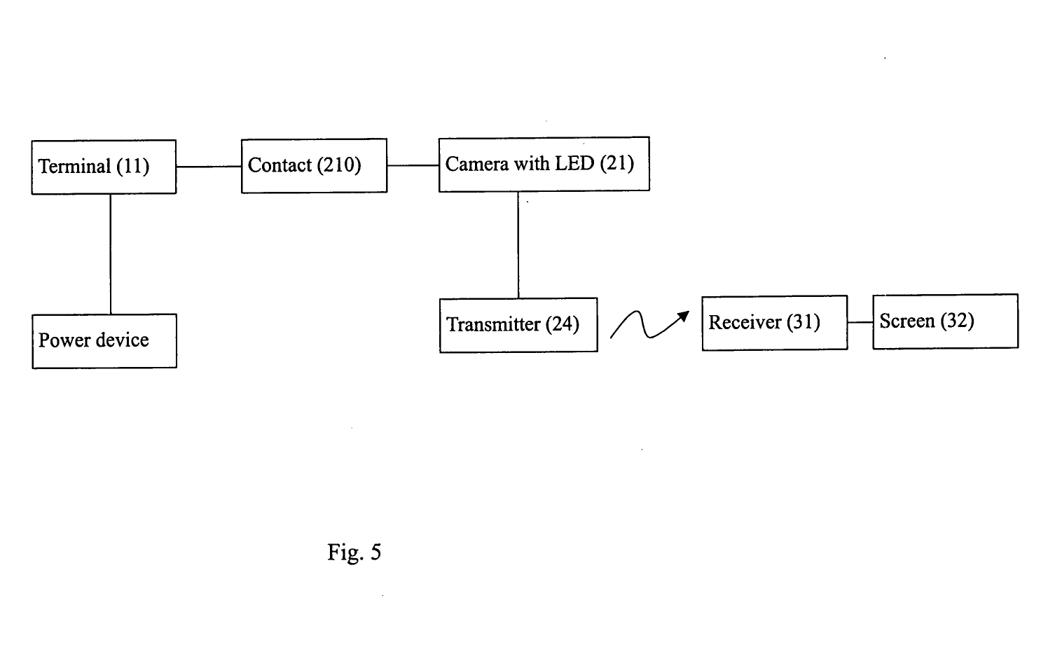

With further reference to FIGS. 2, 3 and 5, the handle (10) has a power device, a terminal (11), a C-shaped block (12), a pin (13) and an optional holding bracket (14). The power device, which can be a battery, is in the handle (10) and can be a battery. The terminal (11) is connected to the power device. The block (12) is formed around the terminal (11). The block (12) has two holes (120), defined through two sides of the block (12). The pin (13) is inserted into the holes (120) of the block (12). The holding bracket (14) has a hook mounted rotatably on an end of the holding bracket (14), and the hook of the holding bracket (14) is mounted on a side of the handle (10). A chamber (140) is defined in the holding bracket (14).

The blade (20) is connected to the handle (10) and has a connecting bracket (201), a depressor (202), a camera with LED (21) and a transmitter (24). The connecting bracket (201) is L-shaped, is mounted in the block (12) and has a notch (22), an inclined surface (23) and an opening (203). The notch (22) is formed in a front end of the connecting bracket (201) and selectively engages the pin (13) of the handle (10). The inclined surface (23) is formed on the front end of the connecting bracket (201) so that the blade (20) can pivotally move to lean against the handle (10). The opening (203) is defined in a rear end of the connecting bracket (201) for the user to see the throat. Hence, the pin (13) can disengage from the notch (22) by horizontal pushing force to detach the handle (10) from the blade (20). The depressor (202) is formed on the front end of the connecting bracket (201) and is curved. The camera with LED (21) is mounted adjacent to the depressor (202) and can be adjusted to different angles. A contact (210) is formed on a top surface of the camera with LED (21) and electrically connects to the terminal (11) to operate the camera with LED (21). The transmitter (24) is mounted on the connecting bracket (201) and connects to the camera with LED (21) to send the image wirelessly. The camera may be a CCD-based (Charge Coupled Device) camera or a CMOS-based (Complementary Metal-Oxide Semiconductor) camera.

The display (30) is mounted in the chamber (140) in the holding bracket (14) and has a receiver (31) mounted in a top end of the screen (30). The receiver (31) can receive and transform the image sent by the transmitter (24) to be shown on the screen (32).

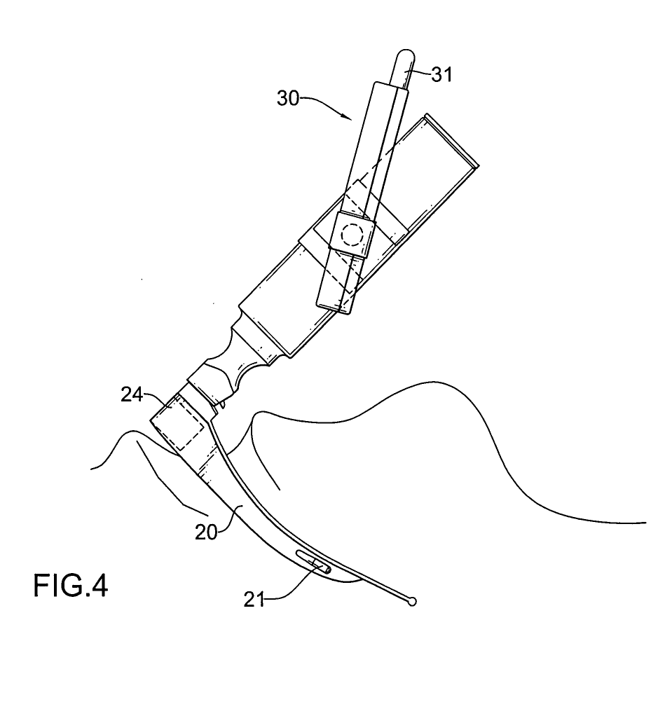

With further reference to FIG. 4, the blade (20) is inserted into the mouth of the patient to lean against the patient's tongue. The throat can be lighted by the camera with LED (21) and the image can be transferred to the display (30). Because the camera with LED (21) is adjustable, the visual angle is increased to provide higher success probability for intubation. Furthermore, the wireless transmission between the transmitter (24) and the screen (30) simplifies the structure of the present invention.

It is to be understood, however, that even though numerous characteristics and advantages of the present invention have been set forth in the foregoing descriptions, together with details of the structure and function of the invention, the disclosure is illustrative only. Changes may be made in details, especially in matters of shape, size, and arrangement of parts within the principles of the invention to the full extent indicated by the broad general meaning of the terms in which the appended claims are expressed.

What is claimed is:

1. A laryngoscope with wireless image transmission comprising:
a handle having
a power device mounted in the handle; and
a terminal formed on the handle and electrically connected to the power device; and
a blade mounted detachably on the handle and having
a connecting bracket mounted detachably on the handle and having a contact formed on the connecting bracket and electrically connected to the terminal of the handle;
a depressor attached to the connecting bracket;
a camera with LED mounted adjacent to the depressor; and
a wireless transmitter electrically connecting to the camera with LED to send images.

2. The laryngoscope with wireless image transmission as claimed in claim 1 further comprising a display, wherein the display has
a receiver receiving and transforming the image sent by the transmitter; and
a screen electrically connected to the receiver to show the image.

3. The laryngoscope with wireless image transmission as claimed in claim 1, wherein
the handle has
a block formed around the terminal; and
a pin inserted into the block; and
the connecting bracket of the blade has a notch formed in a front end of the connecting bracket and selectively engaging the pin of the handle.

4. The laryngoscope with wireless image transmission as claimed in claim 3, wherein the connecting bracket has an inclined surface formed on the front end of the connecting bracket.

5. The laryngoscope with wireless image transmission as claimed in claim 2, wherein
the handle has a holding bracket mounted in a side of the handle and having a chamber defined in the holding bracket; and
the display is mounted in the chamber in the holding bracket.

6. The laryngoscope with wireless image transmission as claimed in claim 5, wherein the holding bracket is mounted pivotally on the handle

7. The laryngoscope with wireless image transmission as claimed in claim 1, wherein the power device in the handle is a battery.
